(19) United States
(12) Patent Application Publication
(10) Pub. No.: US 2006/0148737 A1
(43) Pub. Date: Jul. 6, 2006

(54) Wound healing method and kits

(75) Inventor: John W Harmon, Baltimore, MD (US)

Correspondence Address: BANNER & WITCOFF, 1001 G STREET N W, SUITE 1100, WASHINGTON, DC, 20001, US

(73) Assignee: The John Hopkins University, Baltimore, MD (US)

(21) Appl. No.: 10/540,934

(22) PCT Filed: Dec. 29, 2003
(86) PCT No.: PCT/US03/41437

§ 371 Date: Feb. 24, 2006

Related U.S. Application Data
(60) Provisional application No. 60/437,392, filed on Dec. 31, 2002.
(60) Provisional application No. 60/471,829, filed on May 20, 2003.

(51) Int. Cl. A61K 48/00; A61N 1/00

(52) U.S. Cl. 514/44; 607/2

(57) ABSTRACT

Electroporation is used to enhance the wound-healing benefit provided by transfection of nucleic acids that encode cellular growth factors. Wounds which are amenable to the method include inter alia cutaneous lesions, muscular lesions, osseus lesions, burn wounds, and gastrointestinal anastamoses. Kits comprise electrodes and nucleic acids encoding cellular growth factors.

DNA required to produce a clinical effect and significantly improve the therapeutic impact.

Electroporation has been commonly used for the delivery of DNA to cells in vitro since the early 1980's.⁷ Electroporation is the application of an electrical field across cells in order to increase the permeability of the cell membranes and allow the entry of macromolecules.⁸ The applied electrical field increases transmembrane voltage potential, exceeding membrane dielectric strength, and causing membrane defects through which the charged polynucleotide may pass.⁹ The electrophoretic effect of the field may also enhance DNA migration within tissues.¹⁰ In vivo electroporation has been used to increase intracellular delivery of agents such as chemotherapeutics both directly into tumors and also to enhance transdermal drug delivery.⁹ Although most commonly used for in vitro transfection applications, electroporation has been of benefit in in vivo settings as well.

Improvements in the transfection of liver,^{11,12} muscle,^{13,14} tumour,^{15,16} and cutaneous tissue¹⁷⁻²⁰ have all recently been demonstrated using electroporation. The prior skin experiments, however, were carried out on normal unwounded skin with the clinical goal of immunization.²¹ The efficacy of this technique in abnormal, injured skin for use in wound healing has not been previously reported. There is a need in the art for methods of treatment for improving the healing of wounds.

### BRIEF SUMMARY OF THE INVENTION

In a first embodiment of the invention a method is provided to promote wound healing in a patient. A nucleic acid encoding a growth factor is administered to a patient at a wound site. An electric field is applied to the wound site in an amount sufficient to increase expression of the encoded growth factor.

In a second embodiment a method is provided to promote wound healing in a patient. A nucleic acid encoding HIP 1-α is administered to a patient at a wound site. Between 1 and 20 pulses of between 500 and 2,000 V/cm and between 10 and 1000 microseconds is applied to the wound site. Wound healing is thereby stimulated.

A third embodiment of the invention is a kit for treating wounds. The kit comprises a nucleic acid encoding a growth factor and one or more electrodes for applying an electric field to a wound.

### BRIEF DESCRIPTION OF THE DRAWINGS

FIG. 1 shows a Luciferase plasmid dose response curve in un-electroporated skin tissue.

FIG. 2 shows that naked plasmid injection (10 μg) was found to be superior to either lipofection or polyfection.

FIG. 3A shows the effect of increasing the electroporative amplitude on the relative luciferase activity, at several concentrations of plasmid solution. 6 pulses of the indicated voltage were applied, each with duration of 100 μs and an interval of 125 ms (*=p<0.05 compared to the unelectroporated group).

FIG. 3B shows that six pulses at 1750 v/cm were more effective than 18 pulses with the same characteristics (*=p<0.05 compared to the unelectroporated group).

FIG. 3C shows that a high voltage, short duration (1750 V/cm, 100 μ s) series of 6 pulses is more efficacious than low voltage longer duration (400 V/cm, 20 ms) pulses.

FIG. 3D shows that six low voltage (200 V/cm), long duration (20 ms) caused a 20 fold increase in Luciferase activity in skeletal muscle tissue (*=p<0.01 compared to the unelectroporated group).

FIG. 4 shows the effect of increasing the plasmid load on the efficacy of electroporation. 6×100 μs, 1750 V/cm pulses were given with an interval of 125 ms (*=p<0.05 compared to the unelectroporated group).

FIG. 5 shows serially acquired bioluminescent images of a single mouse after 50 μg injections of luciferase plasmid. Only the wounds on the right side of the animal were electroporated. Images were taken on days 1, 7 and 14.

FIG. 6 shows a time course of luciferase activity after a single naked plasmid injection, with and without electroporation, compared to the unelectroporated group.

FIG. 7A shows day 7 wound areas in electroporated and unelectroporated wounds.

FIG. 7B shows day 7 wound breaking strengths in electroporated and unelectroporated wounds.

FIG. 8 shows a schematic design of pin electrode.

FIG. 9 shows a square wave electroporation characteristics.

FIG. 10 shows the wound areas at day 10 showing that electroporation increased the efficacy of the HIF 1-alpha plasmid expression vector's ability to hasten healing of cutaneous wounds. # P=0.053 vs control group. *p<.0.05 vs control and vs un-electroporated group.

### DETAILED DESCRIPTION OF THE INVENTION

It is a discovery of the present inventor that electroporation enhances the efficiency of transfection by cells at or near a wound site in the body. A nucleic acid encoding at least one growth factor promotes wound healing on its own; electroporation enhances that effect.

Any nucleic acid can be used which encodes a growth factor. Suitable growth factors include but are not limited to Keratinocyte Growth Factor-1, Platelet Derived Growth Factor, Vascular Epidermal Growth Factor, and Hypoxia Induced Factor 1-α. Other suitable growth factors include human EGF, human EG-VEGF, human Erythropoietin, human GDF-11, human Growth Hormone Releasing Factor, human HGF, human KGF, human LCGF, human LIF, human Myostatin, human Oncostatin M, human SCF, human Thrombopoietin, and human VEGF. Still other which can be used include human angiogenesis proteins including: human ACE, human Angiogenin, human Angiopoietin, and human Angiostatin; human bone morphogenetic proteins including: human BMP-13/CDMP-2, human BMP-14/CDMP-1, human BMP-2, human BMP-3, human BMP-4, human BMP-5, human BMP-6, and human BMP-7; human colony stimulating factors including: human flt3-Ligand, human G-CSF, human GM-CSF, and human M-CSF; human fibroblast growth factors including: human FGF-10, human FGF-16, human FGF-17, human FGF-18, human FGF-19, human FGF-20, human FGF-4, human FGF-5, human FGF-6, human FGF-8, human FGF-9, human FGF-acidic, and human FGF-basic; human IGF including: human IGF-I, and human IGF-II; human PDGF including: human PDGF (AA Homodimer), human PDGF (AB Heterodimer), and human PDGF (BB Homodimer); human PIGF including: human PIGF-1, and human PIGF-2; human stem cell growth factors including: human SCGF-alpha, and human SCGF-beta; human transforming growth factors: human TGF-alpha, and human TGF-beta. While human growth factors are listed above, non-human growth factors can be used, particularly when the patient is a non-human animal.

The nucleic acids encoding the growth factors may be in a plasmid or viral vector, or other vector as is known in the art. Such vectors are well known and any can be selected for a particular application. In one embodiment of the invention, the gene delivery vehicle comprises a promoter and a growth factor coding sequence. Preferred promoters are tissue-specific promoters and promoters which are activated by cellular proliferation, such as the thymidine kinase and thymidylate synthase promoters. Other preferred promoters include promoters which are activatable by infection with a virus, such as the α- and β-interferon promoters, and promoters which are activatable by a hormone, such as estrogen. Other promoters which can be used include the Moloney virus LTR, the CMV promoter, and the mouse albumin promoter.

In another embodiment, naked growth factor polynucleotide molecules are used as gene delivery vehicles, as described in WO 90/11092 and U.S. Pat. No. 5,580,859. Such gene delivery vehicles can be either growth factor DNA or RNA and, in certain embodiments, are linked to killed adenovirus. Curiel et al., Hum. Gene. Ther. 3:147-154, 1992. Other vehicles which can optionally be used include DNA-ligand (Wu et al, J. Biol. Chem. 264:16985-16987, 1989), lipid-DNA combinations (Felgner et al., Proc. Natl. Acad. Sci. USA 84:7413 7417, 1989), liposomes (Wang et al., Proc. Natl. Acad. Sci. 84:7851-7855, 1987) and microprojectiles (Williams et al., Proc. Natl. Acad. Sci. 88:2726-2730, 1991).

A growth factor gene delivery vehicle can optionally comprise viral sequences such as a viral origin of replication or packaging signal. These viral sequences can be selected from viruses such as astrovirus, coronavirus, orthomyxovirus, papovavirus, paramyxovirus, parvovirus, picornavirus, poxvirus, retrovirus, togavirus or adenovirus. In a preferred embodiment, the growth factor gene delivery vehicle is a recombinant retroviral vector. Recombinant retroviruses and various uses thereof have been described in numerous references including, for example, Mann et al., Cell 33:153, 1983, Cane and Mulligan, Proc. Nat'l. Acad. Sci. USA 81:6349, 1984, Miller et al., Human Gene Therapy 1:5-14, 1990, U.S. Pat. Nos. 4,405,712, 4,861,719, and 4,980,289, and PCT Application Nos. WO 89/02,468, WO 89/05,349, and WO 90/02,806. Numerous retroviral gene delivery vehicles can be utilized in the present invention, including for example those described in EP 0,415,731; WO 90/07936; WO 94/03622; WO 93/25698; WO 93/25234; U.S. Pat. No. 5,219,740; WO 9311230; WO 9310218; Vile and Hart, Cancer Res. 53:3860-3864, 1993; Vile and Hart, Cancer Res. 53:962-967, 1993; Ram et al., Cancer Res. 53:83-88, 1993; Takamiya et al., J. Neurosci. Res. 33:493-503, 1992; Baba et al., J. Neurosurg. 79:729-735, 1993 (U.S. Pat. No. 4,777,127, GB 2,200,651, EP 0,345,242 and WO91/02805).

A growth factor polynucleotide of the invention can also be combined with a condensing agent to form a gene delivery vehicle. The condensing agent may be a polycation, such as polylysine, polyarginine, polyornithine, protamine, spermine, spermidine, and putrescine. Many suitable methods for making such linkages are known in the art (see, for example, Ser. No. 08/366,787, filed Dec. 30, 1994).

In an alternative embodiment, a growth factor polynucleotide is associated with a liposome to form a gene delivery vehicle. Liposomes are small, lipid vesicles comprised of an aqueous compartment enclosed by a lipid bilayer, typically spherical or slightly elongated structures several hundred Angstroms in diameter. Under appropriate conditions, a liposome can fuse with the plasma membrane of a cell or with the membrane of an endocytic vesicle within a cell which has internalized the liposome, thereby releasing its contents into the cytoplasm. Prior to interaction with the surface of a cell, however, the liposome membrane acts as a relatively impermeable barrier which sequesters and protects its contents, for example, from degradative enzymes. Additionally, because a liposome is a synthetic structure, specially designed liposomes can be produced which incorporate desirable features. See Stryer, Biochemistry, pp. 236-240, 1975 (W. H. Freeman, San Francisco, Calif.); Szoka et al., Biochim. Biophys. Acta 600:1, 1980; Bayer et al., Biochim. Biophys. Acta. 550:464, 1979; Rivnay et al., Meth. Enzymol. 149:119, 1987; Wang et al., PROC. NATL. ACAD. SCI. U.S.A. 84: 7851, 1987, Plant et al., Anal. Biochem. 176:420, 1989, and U.S. Pat. No. 4,762,915. Liposomes can encapsulate a variety of nucleic acid molecules including DNA, RNA, plasmids, and expression constructs comprising growth factor polynucleotides such those disclosed in the present invention.

Liposomal preparations for use in the present invention include cationic (positively charged), anionic (negatively charged) and neutral preparations. Cationic liposomes have been shown to mediate intracellular delivery of plasmid DNA (Felgner et al., Proc. Natl. Acad. Sci. USA 84:7413-7416, 1987), mRNA (Malone et al., Proc. Natl. Acad. Sci. USA 86:6077-6081, 1989), and purified transcription factors (Debs et al., J. Biol. Chem. 265:10189-10192, 1990), in functional form. Cationic liposomes are readily available. For example, N[1-2,3-dioleyloxy)propyl]-N,N,N-triethylammonium (DOTMA) liposomes are available under the trademark Lipofectin, from GIBCO BRL, Grand Island, N.Y. See also Felgner et al., Proc. Natl. Acad. Sci. USA 91: 5148-5152.87, 1994. Other commercially available liposomes include Transfectace (DDAB/DOPE) and DOTAP/DOPE (Boerhinger). Other cationic liposomes can be prepared from readily available materials using techniques well known in the art. See, e.g., Szoka et al., Proc. Natl. Acad. Sci. USA 75:4194-4198, 1978; and WO 90/11092 for descriptions of the synthesis of DOTAP (1,2-bis(oleoyloxy)-3-(trimethylammonio)propane) liposomes.

Similarly, anionic and neutral liposomes are readily available, such as from Avanti Polar Lipids (Birmingham, Ala.), or can be easily prepared using readily available materials. Such materials include phosphatidyl choline, cholesterol, phosphatidyl ethanolamine, dioleoylphosphatidyl choline (DOPC), dioleoylphosphatidyl glycerol (DOPG), dioleoylphoshatidyl ethanolamine (DOPE), among others. These materials can also be mixed with the DOTMA and DOTAP starting materials in appropriate ratios. Methods for making liposomes using these materials are well known in the art.

One or more growth factors may be encoded by a single nucleic acid delivered. Alternatively, separate nucleic acids may encode different growth factors.

Different species of nucleic acids may be in different forms; they may use different promoters or different vectors or different delivery vehicles. Similarly, the same growth factor may be used in a combination of different forms.

Wounds which are amenable to treatment according to the present invention are those on the surface as well as internal to an animal body. Such wounds include but are not limited to cutaneous wounds, muscular wounds, osseus lesions, gastrointestinal anastamoses, decubitus ulcers, gastrointestinal ulcers, and burn wounds. The method of the present invention can be applied to any mammal, including humans, horses, sheep, primates such as monkeys, apes, gibbons, chimpanzees, rodents such as mice, rats, guinea pigs, hamsters, ungulates such as cows.

An electric field to be applied may be of a field strength of 10 to 5,000 V/cm. Suitable ranges include from 10 to 100, from 100 to 500, from 500 to 1,000, and from 1,000 to 5,000 V/m. The field may be uniform or pulsed. If pulsed, a square wave pulse may optionally be used. If the lesion to be treated is an internal lesion, an endoscope can be used to deliver the electric field locally to the lesion.

Electrodes for use in the present invention may be reusable or disposable. If disposable, the electrodes can be pre-sterilized in a sealed package. They can be made of any metal which is non-reactive and non-toxic in the body. Typical metals for such use include, without limitation, brass, gold, stainless steel. Base metals can be coated or plated with a precious metal such as gold. The shape and size of the electrode can be adapted to the size and body location of the wound to be treated. Typical electrode shapes include, but are not limited to needle, paddle, spatula, right-angle, hook, ballpoint, knife, and disk. A handle for receiving the adapters can advantageously be made of an insulating material to protect the operator. Nucleic acids can be coated on disposable electrodes and prepackaged.

Kits according to the present invention are two or more items that are packaged together in a single container. Kits of the present invention typically contain one or more nucleic acids encoding at least one growth factor and one or more electrodes. The growth factor and electrodes may be separately packaged within the single “kit” container which contains them both. Alternatively, the electrodes may be dipped or impregnated with the nucleic acid, and thus not separately packaged. Nucleic acids may be provided in any form which is convenient, including lyophilized, frozen, or liquid forms. The kit may also contain a handle, specifically designed to receive the electrodes. The kit may also contain an electroporator machine. The electrodes may be pre-sterilized. Instructions for the kit may be provided in paper form as a package insert or label. Alternatively, a reference to an external source, such as an internet website may be provided. Instructions may alternatively be provided on an electronic medium included within the kit.

Thus, embodiments of the invention are disclosed. One skilled in the art will appreciate that the present invention can be practiced with embodiments other than those disclosed. The disclosed embodiments are presented for purposes of illustration and not limitation, and the present invention is limited only by the claims that follow.

### EXAMPLES

We assessed the ability of in vivo electroporation to enhance gene expression. Full thickness cutaneous excisional wounds were created on the dorsum of female mice. A Luciferase encoding plasmid driven by a CMV promoter was injected at the wound border. Following plasmid administration, electroporative pulses were applied to injection sites. Pulse parameters were varied over a range of voltage, duration, and number. Animals were sacrificed at intervals after transfection and the Luciferase activity measured. Application of electric pulses consistently increased Luciferase expression. The electroporative effect was most marked at a plasmid dose of 50 μg, where an approximate 10-fold increase was seen. Six 100 as duration pulses of 1750 V/cm were found to be the most effective in increasing Luciferase activity. High numbers of pulses tended to be less effective than smaller numbers. This optimal electroporation regimen had no detrimental effect on wound healing. Electroporation increases the efficiency of trans gene expression and may have a role in gene therapy to enhance wound healing. A HIF 1-alpha encoding plasmid driven by a CMV promoter was then injected at the wound borders of homozygous diabetic mice and found to accelerate wound healing. The enhanced healing was more pronounced in electroporated animals.

### Example 1

### Luciferase Activity after Injection of Naked Plasmid

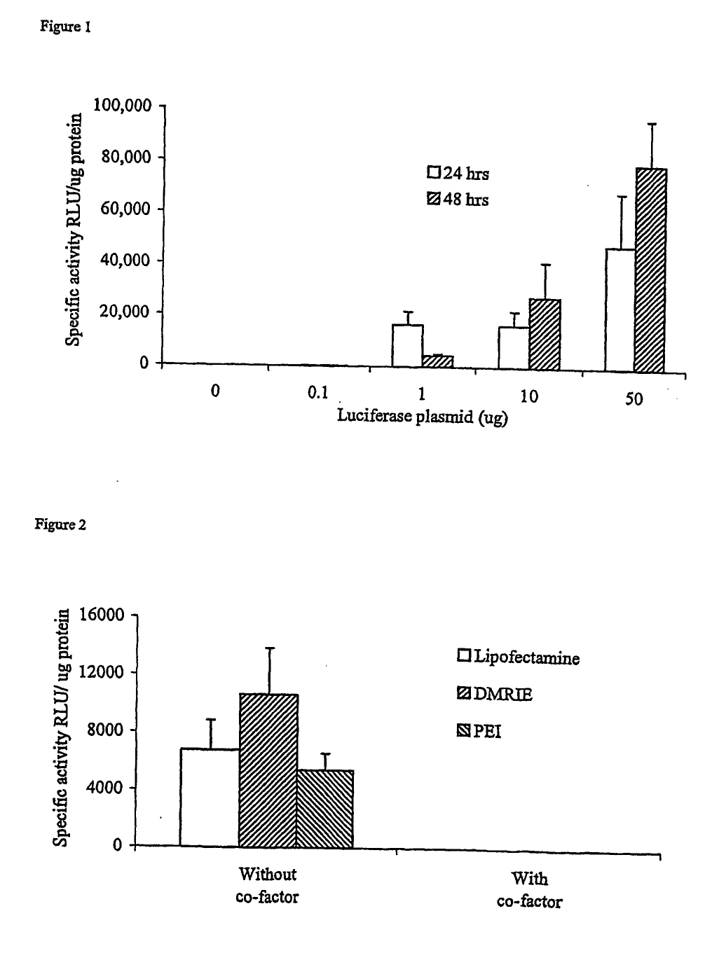

There was no evidence of Luciferase activity in uninjected skin tissue sites. Enzymatic activity was detected at plasmid dosages as low as 0.1 μg plasmid. Increasing the dosage of plasmid injected caused the amount of Luciferase activity to rise across the 500 fold range tested up to 50 μg (FIG. 1).

Lipofection and Polyfection

The addition of Lipofectamine (80 μL/ml), DMRIE (120 μl/ml), or PEI (5:1 ratio of PEI-Nitrogen:DNA-Phosphate) to plasmid solutions consistently reduced or abolished the luciferase activity seen in the skin tissue with 10 μg naked plasmid injection (FIG. 2). The highest luciferase activity was always evident in animals injected with naked plasmid, without either lipofection or polyfection.

### Example 2

### Electroporation Parameters

Voltage Dose Response Effect

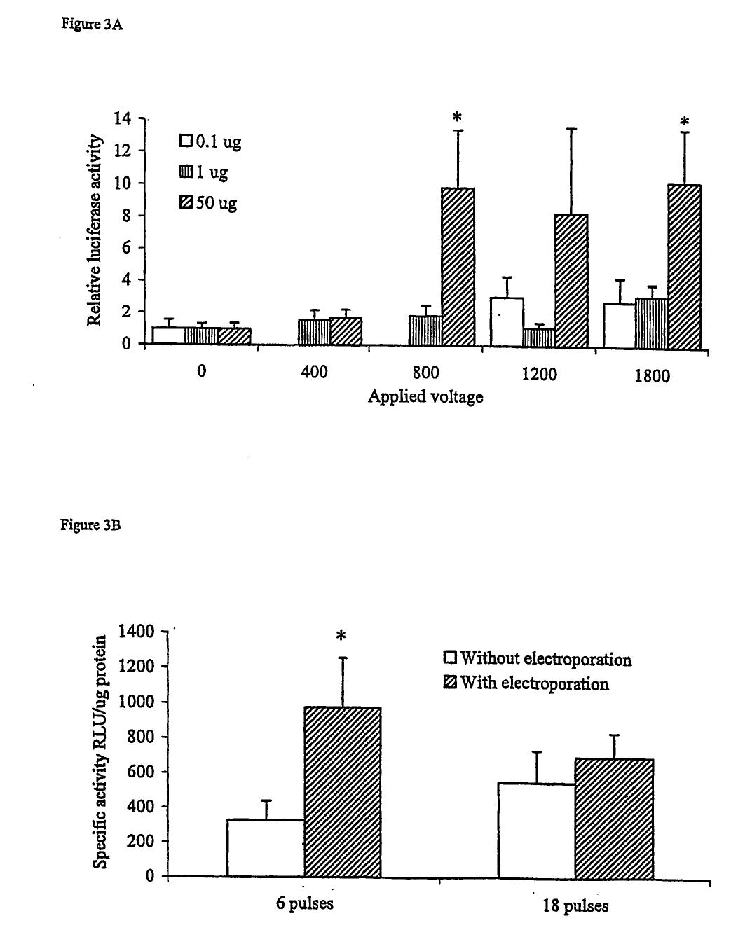

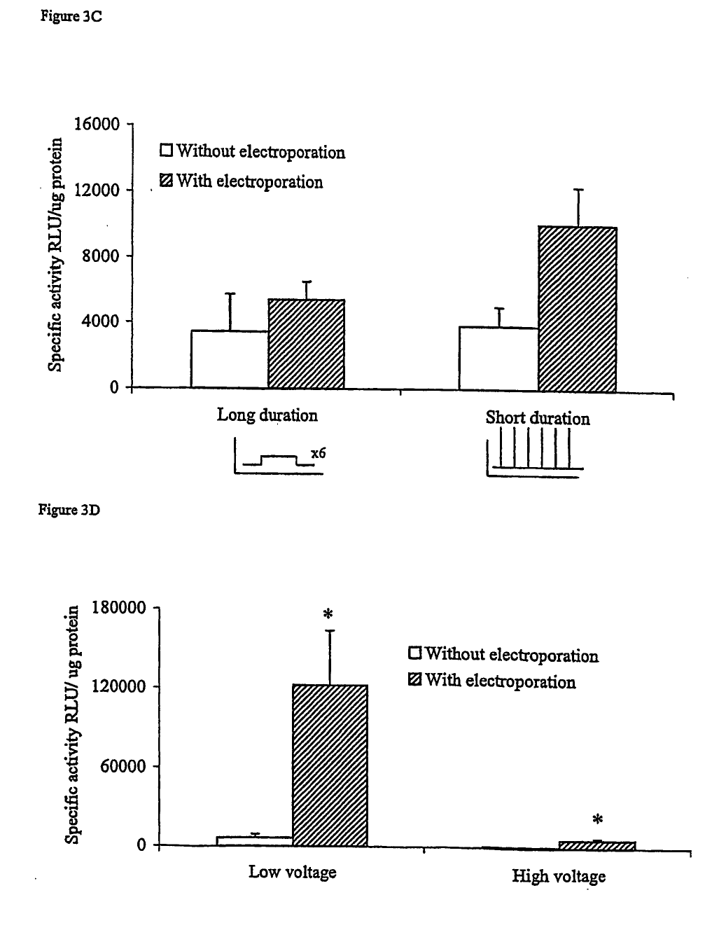

The application of electric pulses locally to the injection site consistently increased the transfection efficiency when measured at 24 hours post injection. Increasing the applied voltage across the injected tissue caused an increase in the Luciferase activity (FIG. 3A). This effect was most apparent at higher plasmid doses of where the increase was over 10 fold. Higher voltages than 1800 V/cm tended to either cause arcing of the electric pulse between the electrodes or left some signs of an electrical burn on the animal's skin.

Pulse Number Effect

Increasing the number of electroporative pulses from 6 to 18 attenuated the increase in transfection efficiency (FIG. 3B).

Pulse Duration Effect

A low voltage long duration series of pulses (6×20 ms, 400 V/cm) was not particularly effective in increasing transfection efficiency with 10 μg plasmid, when compared to high voltage short duration pulses (6×100 μs, 1750 V/cm) FIG. 3C. This is in contrast to skeletal muscle tissue with 10 μg plasmid, where low voltage electroporation parameters caused a 20-fold increase in transfection efficiency (FIG. 3D).

### Example 3

### Plasmid Dose Response Effect with Optimal Electroporation Parameters

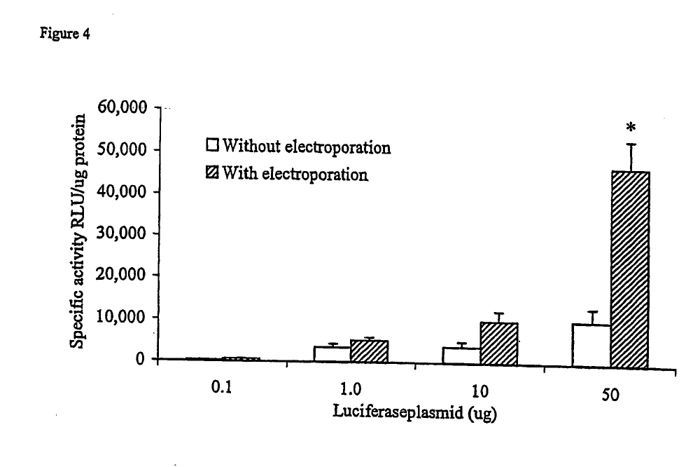

Using the optimal electroporation parameters, the electroporative effect was seen over a range of plasmid doses tested, but was most effective at higher doses of DNA. Using 50 μg of DNA, electroporation produced a large increase in Luciferase activity. With electroporation, 10 μg of plasmid produced luciferase expression equivalent to that achieved with 50 μg of naked plasmid without electroporation (FIG. 4).

### Example 4

### Duration of Transfection

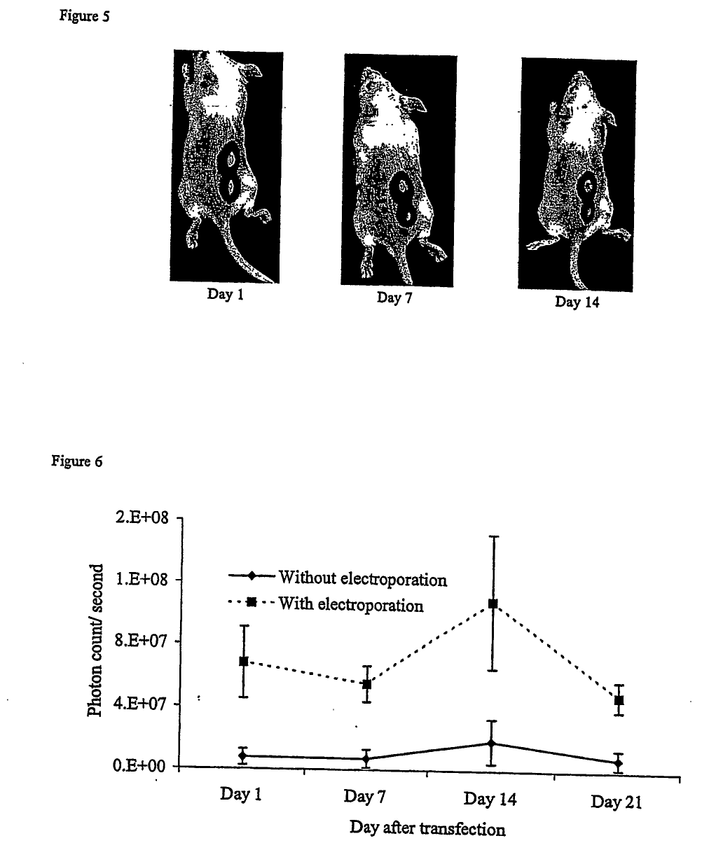

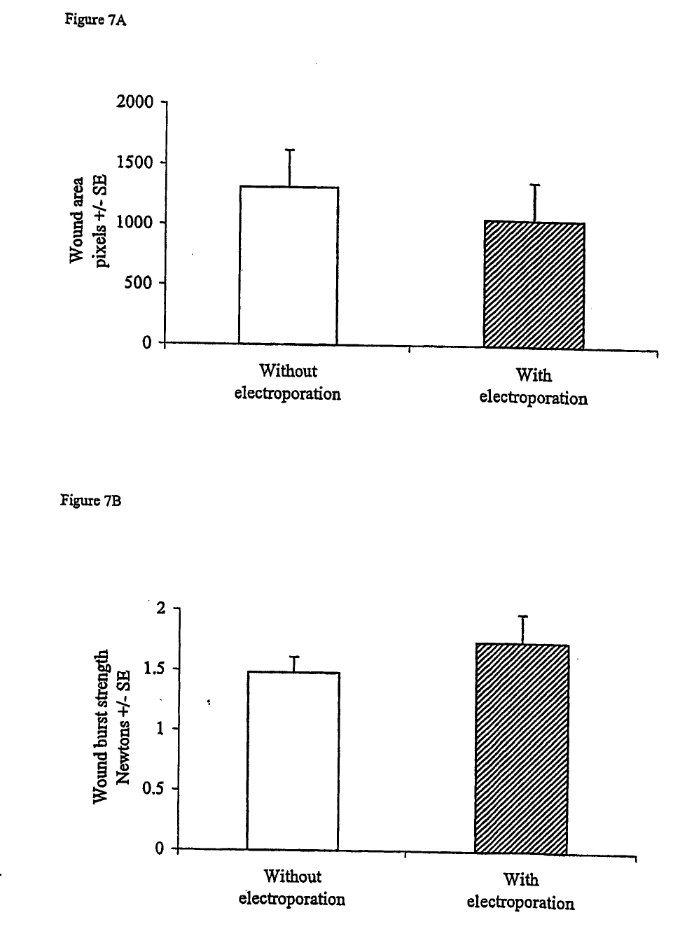

Electroporation of the skin tissue consistently led to an increase in the transfection efficiency after a single injection of plasmid (FIG. 5). In order to examine if electroporation had any effect on the duration of gene expression, animals were imaged at varying intervals after a single plasmid injection, between one day and three weeks (FIG. 6). The Luciferase activity was approximately 10 fold higher (7.71×106+5.24×106 vs. 6.82×107+2.28×107, p<0.01 at day 1) in the electroporated injection sites than in the non-electroporated sites. This increased activity was maintained throughout the duration of the experiment, up to an interval of three weeks.

### Example 5

### Effect of Electroporation on Wound Healing

Measurement of both the wound areas and wound breaking strength at day 7 in animals with and without the administration of the most effective electroporation settings (6×100 μs, 1750 V/cm), had no detrimental effect on these healing parameters. In fact there was a slight non-significant tendency for the electroporated wounds to have improved healing as evidenced by a smaller open area and greater tensile strength (FIGS. 7A and 7B).

### Example 6

### Effect of HIF 1-alpha Plasmid with and without Electroporation on Wound Healing

When the expression vector for HIP 1 alpha was injected into the wound edges at the time of wounding in diabetic mice, there was a significant reduction in wound size at day 10 showing increased healing. The mean wound area determined using the digital imaging system tended to be reduced from 1057±265 to 351±108 pixels, p=0.053. When electroporation (6×100 μs, 1800 V/cm) was added a further significant increment in enhanced wound healing was seen, for those animals which received both HIF 1-alpha and electroporation all wounds had totally healed by day 10 with wound size decreasing from 351±108 to 0, P<0.05.

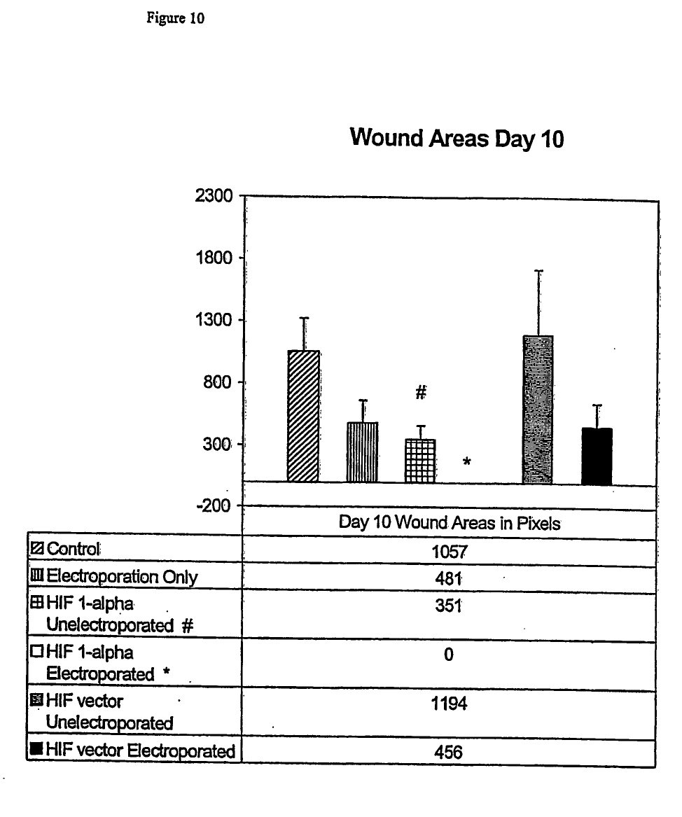

In control groups the vector without the HIF 1-alpha insert did not improve wound healing. Electroporation with or without the empty plasmid vector had a tendency to improve wound healing with a smaller wound seen at day 10 in comparison to the un-electroporated animals. When burst strength was measured at day 14 there were no significant differences between the groups (FIG. 10).

### Example 7

### Materials and Methods

Plasmids

The Plasmid gWIZ-Lux, containing a CMV promoter and luciferase transgene, was obtained from Gene Therapy Systems (San Diego, Calif.). The pCEP4 plasmid with the HIF 1-alpha insert and a CMV promoter was a gift from Dr. Gregory Semenza, Johns Hopkins University, Baltimore Md. Plasmids were purified using an endotoxin free plasmid purification kit (Qiagen, Santa Clarita, Calif.) following culture in transformed DH-5α bacteria. Plasmids were stored at −70° C. at a concentration of 2 mg/ml until use. Lipofectamine and DMRIE-C were obtained from Gibco BRL (Carlsbad, Calif.). Polyethylenimine (PEI) was obtained from Sigma-Aldrich (St. Louis, Mo.).

Plasmid Administration

Female 6-8 week old BALB-c and BKS.Cg-m Lepr^{db/db} (homozygous diabetic) mice were obtained from Jackson Laboratories (Bar Harbor, Me.). All procedures were approved by the Johns Hopkins University Animal Care and Use Committee. Animals were anesthetized with an intraperitoneal injection of 0.02 ml/g of a 1.25% Avertin solution. Their dorsum was shaved and two symmetrical full thickness excisional wounds were created on their backs on both left and right sides using a 5 mm punch biopsy instrument or in the case of diabetic mice with a 4 mm punch biopsy instrument. 50 μL of the appropriate concentration of Luciferase plasmid was injected intradermally both anterior and posterior to each wound. In diabetic animals 10 μg of appropriate plasmid in 50 μL of media was injected anteriorly as well as posteriorly in the wound edges on both sides. The resulting skin blebs confirmed intradermal delivery of the plasmid and were marked with indelible ink. Wounds were left undressed and animals were housed individually.

Electroporation

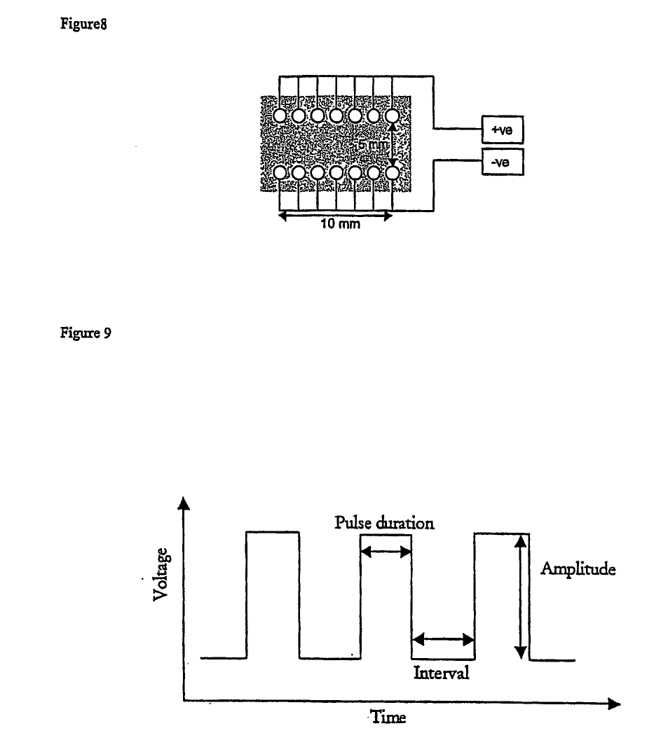

Animals were electroporated at the site of injection within two minutes of plasmid administration, using a square wave electroporator (ECM 830, BTX Genetronics, San Diego, Calif.). A custom designed pin electrode, consisting of two 10 mm rows of parallel needles separated by 5 mm was used to apply the electroporation voltage (FIG. 8). Between 6 to 18 square wave pulses were administered, at an amplitude of between 400 and 1800 volts, a duration of between 100 μs to 20 ms, and an interval between pulses of 125 ms (FIG. 9).

In vitro Luciferase Assay

After at least 24 hours, animals were sacrificed, and 25 mm2 specimens at the marked injection sites were excised. The skin tissue was homogenized in a cell lysis buffer (Pharmingen, San Diego, Calif.) containing a proteinase inhibitor cocktail (Sigma, St. Louis, Mo.), using a polytron homogenizer. Samples were centrifuged at 14,000 RPM for 30 seconds before use. The luciferase activity of each sample was determined using a commercial luciferase assay kit (Pharmingen, San Diego, Calif.). 40 μL of each sample was placed into a luminometer (Mono light 3010, BD Biosciences, San Jose, Calif.) with 100 μL of co-factor solution. 100 μL luciferase substrate was added and the photon emission measured over the following 10 seconds. The protein concentration of each sample was determined using a protein assay kit (BioRad, Hercules, Calif.). Light output was normalized to each sample's protein concentration and luciferase activity expressed as RLU/μg protein.

In vivo Luciferase Imaging

To assess the time course of luciferase expression with and without electroporation, animals were analyzed using an in vivo luciferase imaging system. In these experiments, mice were wounded and injected with plasmid as previously described, but only the injection sites on the right side of each animal were electroporated using six 100 μs pulses of 1750 V/cm, with an interval of 125 ms. At time points after the initial transfection, animals were sedated with intraperitoneal Avertin, and then injected intraperitoneally with 150 mg/kg of D-luciferin in water. After a conventional light photograph was taken, bioluminescent images were acquired using a cooled charged coupled device camera (IVIS, Xenogen, Alameda, Calif.). Luminescent images were taken at intervals of between 10 and 40 minutes following luciferin administration, during which time the light emission had been shown to be in a plateau phase. Bioluminescent images were overlaid onto the conventional image of each animal, and the light emission, corrected for background luminescence, was calculated for each injection site using image analysis software (Living Image, Xenogen, Alameda, Calif.). Activities are expressed as total photons per second for equal sized regions of interest at the injection sites.

Wound Healing Measurements

Animals were anesthetized and wounded as previously mentioned. No plasmid was administered, and half the wounds were electroporated with six, 1750 V/cm square wave pulses of 100 μs duration with 125 ms interval. Animals were sacrificed on day 7 following wounding. The wound eschar was carefully removed and the un-epithelialized wound border traced in situ onto clear acetate paper. Images were digitized at 600 dpi (Visioneer Paperport 6000, Visioneer, Frement, Calif.) and wound areas were calculated using image analysis software based on NIH image (Scion Image, Frederick, Md.). Areas were expressed as a pixel count. The dorsal skin was subsequently removed in the plane deep to the panniculus carnosus muscle. Skin strips were cut to according to a 2×0.5 cm template with the wound at the midpoint. Each strip was loaded onto a custom built tensiometer and traction applied at a rate of 10 mm/minute until complete disruption of the wound occurred. The wound burst strength was recorded in Newtons as the peak force across the tissue prior to fracture. In the second series of experiments six groups of BKS.Cg-m Lepr^{db/db} (homozygous diabetic) mice were studied. The groups included control (wounds only, no plasmid), electroporation only (wounds with no plasmid and six, 1800 V/cm square wave pulses of 100 μs duration with 125 ms interval), plasmid expression vector for HIF 1-alpha with 1800 V/cm electroporation, and without electroporation, and plasmid expression vector (pCEP4) without the HIF 1-alpha insert with and without electroporation. On day 10 wound eschar was carefully removed and the un-epithelialized wound border traced in situ onto clear acetate paper. Images were digitized and wound areas were calculated as in the first series of experiments. Wound burst strengths were measured on day 14.

Statistical Analysis

Results were presented as means±SEM. Differences in means between groups were analyzed for significance using Student's t-test or ANOVA as appropriate with Mann-Whitney Rank Sum Test.

### Example 8

### Discussion

These experiments demonstrate that electroporation can improve plasmid transfection efficiency in cutaneous wound tissue. This effect was maximal, over 10-fold, at the higher doses of plasmid administered to the wounds and at greater electroporation voltages. Using a series of high voltage, short duration pulses was found to be superior in efficacy to lower voltage, longer duration pulses. The electroporation protocol was not detrimental to wound healing. Importantly electroporation significantly improved the ability of the growth factor Hypoxia Induced Factor 1-alpha to speed wound closure in our diabetic mouse model. HIF 1-alpha plasmid treatment alone hastened wound closure, with the treated wounds having less than half the open area as the untreated wounds at 10 days. However, with the addition of electroporation, the HIF 1-alpha treated wounds were completely closed by 10 days. This demonstrates the therapeutic efficacy of electroporation to enhance plasmid transfection.

Burst strengths were tested at day 14 at which time the wounds in all the groups had closed. At that time point there were no differences in burst strength among the groups. The effect seen may be of considerable benefit in wound healing applications.

Gene therapy has potential to treat a wide spectrum of both genetic and acquired diseases. The skin may be transfected in gene therapy applications for both systemic treatment, such as immunization, as well as local therapy, including the enhancement of wound healing.²² Ex vivo gene therapy techniques have been used in the field of wound healing,^{23,24} but in vivo techniques have the advantage of being simpler and less time consuming, making them more appropriate for potential clinical use.²⁵ Prior experience in our laboratory and others has shown that the use of DNA plasmids encoding different growth factors can improve wound healing in animal models.²⁶⁻²⁸ The main barrier for in vivo gene therapy is delivery of DNA molecules to tissues in such a manner that they are efficiently expressed.²⁹ The DNA must reach the nucleus to be expressed. Exogenous DNA tends to be sequestered in the extracellular tissue, or in the cell cytoplasm.^{30,31} Viral gene delivery has the advantage of achieving nuclear entry with high transfection efficiencies, particularly in non-dividing cells and in vivo. However there are serious concerns regarding the safety and immunogenicity of current viral mediators. Numerous techniques have been described for non-viral transfection of skin and other tissues, including naked plasmid injection,^{32,33} topical application,³⁴ biolistic delivery with a gene gun35 and microseeding.³⁶ However in vivo transfection efficiency with these techniques remains several orders of magnitude less efficient than that of in vitro transfection. Increasing gene expression with lipofection is effective in serum free tissue culture settings, but not in the tissue setting. The liposomal agents bind to extracellular protein and actually prevent DNA uptake into cells. Interestingly lipofection has been shown to be of some benefit following intraluminal delivery of plasmid into hollow visci, including blood vessels,^{37,38} the lung,³⁹ and colon.⁴⁰ But we found that in skin, the liposomal agents used had a detrimental effect on transfection efficiency when compared to the injection of naked plasmid alone. Prior reports have also suggested that lipofection or polyfection may not be advantageous in skin tissue.^{41,42} It is interesting to compare the effects of electroporation in skin with its effects in other tissues. Muscle seems to be the ideal target for in vivo electroporation. It is suggested that the large size of striated muscle cells gives them properties that interact favorably with an electrical field. Increases in transfection efficiency of 2 to 4 log with relatively low voltage electrical fields have been achieved in striated muscle.¹³ Our results in skin are modest in comparison. We demonstrate that electroporation is a simple, safe, and efficacious means of improving transfection efficiency in skin wounds. The application of high voltage, short duration, square wave electrical field pulses to wounded tissue can enhance gene expression over 10 fold. With this approach the dose of plasmid can therefore potentially be reduced 10 fold as compared to what has been required with naked plasmid. This decrease in the dose of DNA application is important as it is likely to diminish the detrimental effect on wound healing seen with high doses of DNA that we have reported previously.⁶ In combination with one or more appropriate transgene(s) encoding growth factors, electroporation has considerable potential in cutaneous wound healing applications.

### REFERENCES

1. Robson M C, et al. The safety and effect of topically applied recombinant basic fibroblast growth factor on the healing of chronic pressure sores. Ann Surg 1992;216(4):401-6.
2. Steed D L. Clinical evaluation of recombinant human platelet-derived growth factor for the treatment of lower extremity diabetic ulcers. Diabetic Ulcer Study Group. J Vasc Surg 1995;21(1):71-8.
3. Robson M C, et al. Recombinant human platelet-derived growth factor-BB for the treatment of chronic pressure ulcers [see comments] Reduced expression of PDGF and PDGF receptors during impaired wound healing. Ann Plast Surg 1992;29(3): 193-201.
4. Greenhalgh D A, Rothnagel J A, Roop D R. Epidermis: an attractive target tissue for gene therapy. J Invest Dermatol 1994; 103(5 Suppl):63S-9S.
5. Felgner P L, Rhodes G. Gene therapeutics. Nature 1991;349(6307):351-2.
6. Byrnes C K, et al. Success and limitations of a naked plasmid transfection protocol for keratinocyte growth factor-1 to enhance cutaneous wound healing. Wound Repair Regen 2001;9(5):341-6.
7. Neumann E, et al. Gene transfer into mouse lyoma cells by electroporation in high electric fields. EMBO J 1982;1(7):841-5.
8. Somiari S, et al. Theory and in vivo application of electroporative gene delivery. Mol Ther 2000;2(3): 178-87.
9. Prausnitz M R, et al. Electroporation of mammalian skin: a mechanism to enhance transdermal drug delivery. Proc Natl Acad Sci USA 1993;90(22):10504-10508.
10. Satkauskas S, et al. Mechanisms of in Vivo DNA Electrotransfer: Respective Contributions of Cell Electropermeabilization and DNA Electrophoresis. Mol Ther 2002;5(2): 133-40.
11. Heller R, et al. In vivo gene electroinjection and expression in rat liver. FEBS Lett 1996;389(3):225-8.
12. Suzuki T, et al. Direct gene transfer into rat liver cells by in vivo electroporation. FEBS Lett 1998;425(3):436-40.
13. Mir L M, et al. High-efficiency gene transfer into skeletal muscle mediated by electric pulses. Proc Natl Acad Sci USA 1999;96(8):4262-7.
14. Mathiesen I. Electropermeabilization of skeletal muscle enhances gene transfer in vivo. Gene Ther 1999;6(4):508-14.
15. Heller L, et al. Electrically mediated plasmid DNA delivery to hepatocellular carcinomas in vivo. Gene Ther 2000;7(10):826-9.
16. Wells J M, et al. Electroporation-enhanced gene delivery in mammary tumors. Gene Ther 2000;7(7):541-7.
17. Titomirov A V, Sukharev S, Kistanova E. In vivo electroporation and stable transformation of skin cells of newborn mice by plasmid DNA. Biochim Biophys Acta 1991;1088(1):131-4.
18. Glasspool-Malone J, et al. Efficient nonviral cutaneous transfection. Mol Ther 2000;2(2): 140-6.
19. Drabick J J, et al. Cutaneous transfection and immune responses to intradermal nucleic acid vaccination are significantly enhanced by in vivo electropermeabilization. Mol Ther 2001;3(2):249-55.
20. Heller R, et al. Intradermal delivery of interleukin-12 plasmid DNA by in vivo electroporation. DNA Cell Biol 2001;20(1):21-6.
21. Dujardin N, Van Der Smissen P, Pre at V. Topical gene transfer into rat skin using electroporation. Pharm Res 2001;18(1):61-6.
22. Eming S A, Morgan J R, Berger A. Gene therapy for tissue repair: approaches and prospects. Br J Plast Surg 1997;50(7):491-500.
23. Vogt P M, et al. Genetically modified keratinocytes transplanted to wounds reconstitute the epidermis. Proc Natl Acad Sci USA 1994;91(20):9307-11.
24. Rosenthal F M, et al. Paracrine stimulation of keratinocytes in vitro and continuous delivery of epidermal growth factor to wounds in vivo by genetically modified fibroblasts transfected with a novel chimeric construct. In Vivo 1997;11(3):201-8.
25. Vogel J C. Nonviral skin gene therapy. Hum Gene Ther 2000;11(16):2253-9.
26. Andree C, et al. In vivo transfer and expression of a human epidermal growth factor gene accelerates wound repair. Proc Natl Acad Sci USA 1994;91(25): 12188-92.
27. Sun L, et al. Transfection with aFGF cDNA improves wound healing. J Invest Dermatol 1997;108(3):313-8.
28. Eming S A, et al. Particle-mediated gene transfer of PDGF isoforms promotes wound repair. J Invest Dermatol 1999; 112(3):297-302.
29. Zabner J, et al. Cellular and molecular barriers to gene transfer by a cationic lipid. J Biol Chem 1995;270(32):18997-9007.
30. Nishikawa M, Huang L. Nonviral vectors in the new millennium: delivery barriers in gene transfer. Hum Gene Ther 2001;12(8):861-70.
31. Byrnes C K, et al. Novel Nuclear Shuttle Peptide to Increase Transfection Efficiency in Esophageal Mucosal Cells. J Gastrointest Surg 2002; (in press)
32. Wolff J A, et al. Direct gene transfer into mouse muscle in vivo. Science 1990;247(4949 Pt 1):1465-8.
33. Hengge D R, Walker P S, Vogel J C. Expression of naked DNA in human, pig, and mouse skin. J Clin Invest 1996;97(12):2911-6.
34. Yu W H, et al. Topical gene delivery to murine skin. J Invest Dermatol 1999;112(3):370-5.
35. Williams R S, et al. Introduction of foreign genes into tissues of living mice by DNA-coated microprojectiles. Proc Natl Acad Sci USA 1991;88(7):2726-30.
36. Eriksson E, et al. In vivo gene transfer to skin and wound by microseeding. J Surg Res 1998;78(2):85-91.
37. Zhu N, et al. Systemic gene expression after intravenous DNA delivery into adult mice. Science 1993;261(5118):209-11.
38. Liu Y, et al. Cationic liposome-mediated intravenous gene delivery. J Biol Chem 1995;270(42):24864-70.
39. Fortunati E, et al. In vitro and in vivo gene transfer to pulmonary cells mediated by cationic liposomes. Biochim Biophys Acta 1996;1306(1):55-62.
40. Liptay S, et al. Colon epithelium can be transiently transfected with liposomes, calcium phosphate precipitation and DEAE dextran in vivo. Digestion 1998;59(2):142-7.
41. Udvardi A, et al. Uptake of exogenous DNA via the skin. J Mol Med, 1999;77(10):744-50.
42. Meuli M, et al. Efficient Gene Expression in Skin Wound Sites Following Local Plasmid Injection. Nonviral skin gene therapy. J Invest Dermatol, 2001; 116(1): 131-5.

What is claimed is:

1. A method to promote wound healing in a patient, comprising:
administering a nucleic acid encoding a growth factor to a patient at a wound site; and
applying an electric field to the wound site in an amount sufficient to increase expression of the encoded growth factor.

2. The method of claim 1 wherein the electric field is applied in pulses.

3. The method of claim 2 wherein 1 to 100 pulses are applied to the wound site.

4. The method of claim 2 wherein the pulse is from 1 microsecond to 5 seconds in duration.

5. The method of claim 1 wherein the electric field is from 10 to 5,000 V/cm.

6. The method of claim 2 wherein the pulse is a square wave pulse.

7. The method of claim 1 wherein the wound is cutaneous.

8. The method of claim 1 wherein the wound is muscular.

9. The method of claim 1 wherein the wound is an osseus lesion.

10. The method of claim 1 wherein the wound is a gastrointestinal anastamosis.

11. The method of claim 1 wherein the growth factor is Keratinocyte Growth Factor-1 (KGF-1).

12. The method of claim 1 wherein the growth factor is Platelet Derived Growth Factor (PDGF).

13. The method of claim 1 wherein the growth factor is vascular epidermal growth factor (VEGF).

14. The method of claim 1 wherein the growth factor is hypoxia induced factor 1-α (HIF 1-α).

15. The method of claim 1 wherein the wound is a burn wound.

16. The method of claim 1 wherein the electric field is applied via an endoscope.

17. The method of claim 1 wherein the wound is a decubitus ulcer.

18. The method of claim 1 wherein one or more nucleic acids encoding at least two growth factors is administered.

19. The method of claim 1 wherein the nucleic acid is a plasmid.

20. The method of claim 1 wherein the patient is diabetic.

21. The method of claim 1 wherein the wound eschar is removed surgically prior to administering the nucleic acid.

22. A method to promote wound healing in a patient, comprising:
administering a nucleic acid encoding a HIF 1-α to a patient at a wound site; and
applying between 1 and 20 pulses of between 500 and 2,000 V/cm and between 10 and 1000 microseconds to the wound site, whereby wound healing is stimulated.

23. The method of claim 22 wherein the wound eschar is removed surgically prior to administering the nucleic acid.

24. The method of claim 22 wherein the nucleic acid is a plasmid

25. A kit for treating wounds, comprising:
a nucleic acid encoding a growth factor; and
one or more electrodes for applying an electric field to a wound.

26. The kit of claim 25 wherein the electrode is disposable.

27. The kit of claim 25 wherein the electrode is sterile.

28. The kit of claim 25 wherein the electrode is needle-shaped.

29. The kit of claim 25 wherein the electrode is paddle-shaped.

30. The kit of claim 25 wherein the electrode is disk-shaped.

31. The kit of claim 25 wherein the electrode is stainless steel.

32. The kit of claim 25 wherein the electrode is gold-coated.

33. The kit of claim 25 wherein the electrode is gold-plated.

34. The kit of claim 25 wherein the electrode is gold-tipped.

35. The kit of claim 25 wherein the electrode is brass.

36. The kit of claim 25 wherein the electrode is coated with the nucleic acid.

37. The kit of claim 26 further comprising a re-usable handle for receiving the one or more electrodes.

38. The kit of claim 25 wherein the nucleic acid is in a container separate from the one or more electrodes.

39. The kit of claim 25 further comprising an electoporator configured to generate an electric field.

40. The kit of claim 25 further comprising an electroporator configured to generate an electric pulse.
